(19) United States
(12) Patent Application Publication
(10) Pub. No.: US 2021/0015942 A1
(43) Pub. Date: Jan. 21, 2021

(54) ANTIBODY PROTAC CONJUGATES

(71) Applicant: Development Center for Biotechnology, Taipei City (TW)

(72) Inventors: Shih-Hsien Chuang, Taipei City (TW); Chu-Bin Liao, Taipei City (TW); Wei-Ting Sun, Taipei City (TW); Chen-Hsien Liang, Taipei City (TW); Wun-Huei Lin, Taipei City (TW); Chun-Liang Lai, Taipei City (TW); Her-Sheng Lin, Taipei City (TW)

(73) Assignee: Development Center for Biotechnology, Taipei City (TW)

(21) Appl. No.: 16/244,090

(22) Filed: Jan. 9, 2019

Related U.S. Application Data
(60) Provisional application No. 62/615,955, filed on Jan. 10, 2018.

(51) Int. Cl. A61K 47/68; C07K 16/32; A61P 35/00

(52) U.S. Cl. CPC A61K 47/6851; C07K 16/32; A61P 35/00; A61K 47/6803; A61K 47/6849

(57) ABSTRACT

An immunoconjugate having the Formula Ab-[L¹-(A-L²-B)ₘ]ₙ, wherein: (a) Ab is an antibody or a binding fragment thereof; (b) L¹ and L² are each independently a linker, wherein L¹ and L² are the same or different and wherein L¹ links to L²; (c) A is a target-protein ligand/binder; (d) B is a ubiquitin ligase ligand/binder, and (e) n and m are independently integers from 1 to 8. The target protein includes kinase, G protein-coupled receptors, transcription factor, phosphatases, and RAS superfamily members.

### FIELD OF THE INVENTION

The present invention relates to novel therapeutic agents based on ADC and PROTAC technology.

### BACKGROUND OF THE INVENTION

Antibody has long been an integral tool in basic research as well as medical use due to their high specificity and affinity for target antigens. A critical feature of antibody is their high specificities and their abilities to bind target antigens, marking them for removal by complement-dependent cytotoxicity (CDC) or antibody-dependent cell-mediated cytotoxicity (ADCC). Antibodies can also impart therapeutic benefits by binding and inhibiting the function of target antigens. However, many unmodified antibodies against tumor-specific antigens often lack therapeutic activities. Although some antibodies can instead be applied successfully as guided missiles to deliver potent cytotoxic drugs in the form of antibody drug conjugates (ADCs), many ADCs have limited therapeutic potentials and further improvements may be required.

A proteolysis targeting chimera (PROTAC) is a two-headed molecule capable of removing unwanted proteins by inducing selective intracellular proteolysis. PROTACs consist of two protein binding moieties, one for binding an E3 ubiquitin ligase and the other for binding a target protein. By binding both proteins, PROTAC brings the target protein to E3 ligase, resulting in the tagging (i.e., ubiquitination) of the target protein for subsequent degradation by the proteasome.

Ubiquitination involves three main steps: activation, conjugation, and ligation, performed by ubiquitin-activating enzymes (E1s), ubiquitin-conjugating enzymes (E2s), and ubiquitin ligases (E3s), respectively. The result of this sequential cascade is to covalently bind ubiquitin to the target protein. The ubiquitinated proteins eventually get degraded by proteasome.

The PROTAC technology was first described in 2001 (Sakamoto et al., “Protacs: chimeric molecules that target proteins to the Skp1-Cullin-F box complex for ubiquitination and degradation,” Proceedings of the National Academy of Sciences of the United States of America. 98 (15): 8554-9). Since then, this technology has been used in several drug designs: pVHL, MDM2, beta-TrCP1, cereblon, and c-IAP1. While these prior art PROTAC drugs are very useful, there is still a need for better PROTAC drugs.

### SUMMARY

Embodiments of the invention relate to branched Antibody-PROTAC Conjugates (APCs). A branched antibody-PROTAC conjugates of the invention combines the advantages of both the ADC and PROTAC approaches, and the branched form of APCs have several benefits in either development or processing compared to the linear form of APCs. In a branched antibody-PROTAC conjugate of the invention, the payload (drug) in a conventional ADC is replaced with a PROTAC and link to the linker part of a PROTAC molecule. These new therapeutics are highly selective, less toxic, safer to use, and with longer in vivo half-lives.

One aspect of the invention relates to immunoconjugates. An immunoconjugate in accordance with one embodiment of the invention has the Formula (I): Ab-[L²-(A-L¹-B)ₘ]ₙ, wherein: (a) Ab is an antibody or a binding fragment thereof; (b) L¹ and L² are each independently a linker, wherein L¹ and L² may be the same or different; (c) A is a target-protein ligand/binder; (d) B is a ubiquitin ligase ligand/binder, and (e) n and m each are independently an integer from 1 to 8.

In accordance with some embodiments of the invention, the target protein may be kinases, G protein-coupled receptors, transcription factor, phosphatases, and RAS superfamily members.

Other aspect of the invention will become apparent with the following description and the included drawings. APC and the structure of a branched

### BRIEF DESCRIPTION OF THE DRAWINGS

FIG. 1 shows a schematic illustrating the structure of a linear (non-branched) APC and the structure of a branched APC.

FIG. 2 shows promotion of BRD4 degradation by various concentrations of ARV-825 and inventive Compound 5, but not inventive Compound 7, in BT-474 breast cancer cell cultures, as analyzed with SDS-PAGE electrophoresis and western blots. ARV-825 is a known small molecule BRD4-targeting PROTAC. Inventive Compound 5 is a branched form of ARV-825. The result shows that Compound 5 has the comparable degradation activity for BRD4 to ARV-825. In contrast, inventive Compound 7, which is Compound 5 with an additional lysosomally cleavable dipeptide (Valine-Citrulline), cannot degrade BRD4 protein at up to 1 μM of Compound 7 treatment. This result probably arises from the fact that Compound 7, with a relatively lower permeability due to the Valine-Citrulline dipeptide, cannot be efficiently internalized into the cells. BRD4 and AKT mark the locations of the bands of BRD4 and AKT, and actin was used as a loading control.

FIG. 3 shows that Example 1 exhibits a specific BRD4 protein degradation activity in HER2-positive BT-474 breast cancer cells instead of HER2-negative MDA-MB-231 breast cancer cells. Example 1 is a branched trastuzumab-Compound 7 immunoconjugate (i.e., a branched APC with trastuzumab as the antibody and Compound 7 as the PROTAC). Moreover, this branched APC would not cause degradation of either AKT protein or actin protein.

FIG. 4 shows a synthetic scheme for a linear APC, by linking through the A binder in ARV-825.

### DETAILED DESCRIPTION

Embodiments of the invention relate to branched APCs. A branched APC couples an antibody with a PROTAC via the linker part of PROTAC. A branched APC of the invention may be viewed as an analog of an antibody-drug conjugate (ADC), in which the payload (drug) in a conventional ADC is replaced with a PROTAC, which conjugates with the antibody through the linker in the PROTAC. That is, in a branched APC of the invention, an antibody (or a binding fragment thereof) is covalently linked, via a linker (L²), with a PROTAC through the linker part (L¹) of PROTAC, instead of linking trough the target protein binding part (A) or the ubiquitin ligase binder (B). The covalent linkage on the antibody may be on the protein portion (e.g., the constant regions or variable region) or on the carbohydrates (the glyco-part).

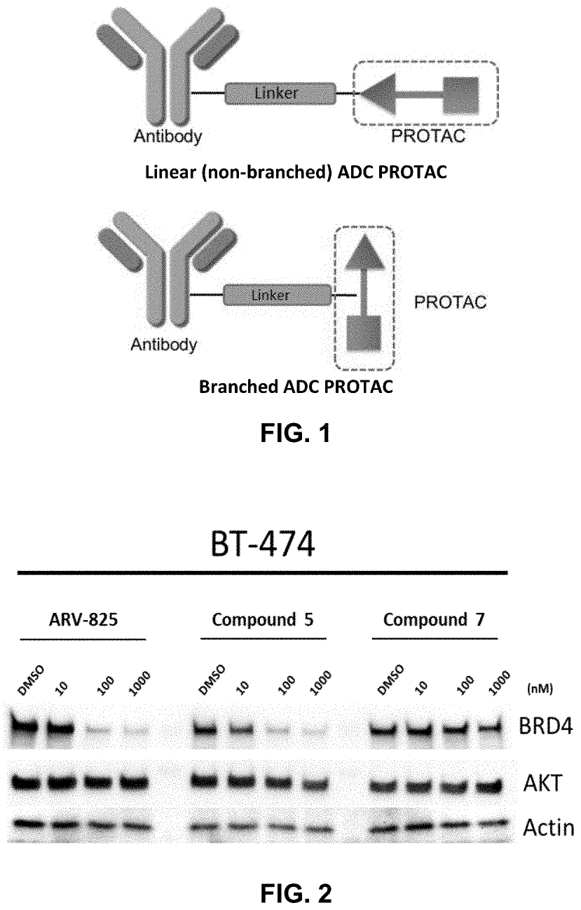

FIG. 1 shows a schematic illustrating the difference between a liner APC and a branched APC. The advantages of branched APCs over linear APCs may include the following:
1. Maintaining the original structure of the target ligand (A) and the E3 ligase ligand (B) in a PROTAC moiety such that the binding affinities of A and B will not change, because the attachment of the antibody is to the linker (L¹) in the PROTAC moiety, instead of at either end (A or B) of the PROTAC moiety.
2. Structure modifications for attaching groups on linkers are much easier than modifications on ligands (A or B), and the attachment between the two linker moieties (L¹ and L²) is more flexible.
3. Modifications on linkers are suitable for most APCs. Therefore, it is possible to design the linkers to use common coupling functional groups such that different antibody moieties may be readily coupled with the same PROTAC or the same antibody may be readily coupled with different PROTACs.

A branched APC in accordance with embodiments of the invention may be represented with the following Formula (I):

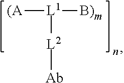

wherein:
(a) Ab is an antibody or a binding fragment thereof;
(b) L¹ and L² are independently linkers, wherein L¹ is a linker within the PROTAC moiety (i.e., PROTAC=A-L¹-B), and L² is a linker that links the antibody with the PROTAC through the linker part (L¹) of PROTAC;
(c) A is a target ligand/binder (i.e., a binder for the target protein, which may be a kinase, a G protein-coupled receptor, a transcription factor, a phosphatase, a RAS superfamily member, etc.)
(d) B is a ubiquitin ligase ligand/binder, wherein the ubiquitin ligase may be E2 or E3 ubiquitin ligase, and
(e) n and m are independently integers from 1 to 8.

The branched APCs of the invention combine the advantages of both ADCs and PROTACs and represent a new class of therapeutics. The term “branched” refers to the structure shown in the above Formula (I), in which the antibody-L² linker is coupled to the L¹ linker in the PROTAC. Other types of APCs, in which the antibody-L² linker is attached to either end (A or B) of the PROTACs, will be referred to as “non-branched” or “linear.” These new branched APCs are highly selective, have long in vivo half-lives, have large therapeutic windows, a broad applicability, and are safer to use. In addition, these branched APCs are easier to synthesize than the non-branched (linear) APCs.

Antibody-drug conjugates (ADCs) are a class of therapeutics, in which a drug (or payload) is attached to an antibody or an antigen-binding fragment thereof. The antibody in an ADC binds to a selected target (typically, a target on a cell), thereby bring the drug to the vicinity of the target, resulting in highly selective therapeutic effects. An example of an ADC may be an antibody targeting a protein expressed on cancer cells, and the payload may be a cytotoxic agent (e.g., Taxol).

ADCs are large molecules due to the presence of an antibody, with molecular weights typically around 150 KDa or more. Thus, ADCs will not be eliminated by kidney filtration. In addition, the antibody constant regions include sites for interactions with receptors in kidney, which can transport and recycle the antibody back into circulation. Therefore, antibodies have long in vivo half-lives, typically several weeks. Furthermore, antibodies can be readily internalized by cells, making delivery of the payloads into the cells very efficient. Because ADCs are specific and long acting, they are promising therapeutic agents. However, the actions of ADCs rely on the payloads, which do not function like catalysts. Therefore, sufficient amounts of payloads are required to kill or suppress the target proteins or cells. Excessive payloads may cause toxicities.

PROTACs consist of two protein-binding moieties, one for binding an E3 ubiquitin ligase and the other for binding a target protein. A PROTAC can bind its target protein and bring it to E3 ubiquitin ligase. Upon tertiary complex formation, E3 ubiquitin ligases transfer ubiquitin to the surface lysines of the target protein, leading to an ubiquitinated target protein that is destined for degradation by the proteasome machinery. After ubiquitination, PROTACs is released and continues to find target protein for ubiquitination and degradation. Thus, PROTACs work like catalysts, and a small amount of PROTACs can achieve substantial results.

In the above Formula (I), the A component is a group that binds to a target protein intended to be degraded. The A component may include any moiety which binds to the target protein specifically. The following are non-limiting examples of small molecule target protein-binding moieties: Hsp90 inhibitors, kinase inhibitors, MDM2 inhibitors, compounds targeting Human BET Bromodomain-containing proteins, HDAC inhibitors, human lysine methyltransferase inhibitors, angiogenesis inhibitors, immunosuppressive compounds, and compounds targeting the aryl hydrocarbon receptor (AHR), among others. The compositions described below exemplify some of the members of these types of small molecule target protein-binding moieties. Such small molecule target protein-binding moieties also include pharmaceutically acceptable salts, enantiomers, solvates and polymorphs of these compositions, as well as other small molecules that may target a protein of interest.

In general, target proteins may include, for example, structural proteins, receptors, enzymes, cell surface proteins, proteins pertinent to the functions of a cell, etc. Accordingly, the A component of a ADC-PROTAC may be any peptide or small molecule that binds protein targets, such as FoxO1, HDAC, DP-1, E2F, ABL, AMPK, BRK, BRSK I, BRSK2, BTK, CAMKK1, CAMKK alpha, CAMKK beta, Rb, Suv39HI, SCF, p19INK4D, GSK-3, pi8 INK4, myc, cyclin E, CDK2, CDK9, CDG4/6, Cycline D, p16 INK4A, cdc25A, BMI1, SCF, Akt, CHK1/2, C 1 delta, CK1 gamma, C 2, CLK2, CSK, DDR2, DYRK1A/2/3, EF2K, EPH-A2/A4/B 1/B2/B3/B4, EIF2A 3, Smad2, Smad3, Smad4, Smad7, p53, p21 Cipl, PAX, Fyn, CAS, C3G, SOS, Tal, Raptor, RACK-1, CRK, Rapl, Rac, KRas, NRas, HRas, GRB2, FAK, PI3K, spred, Spry, mTOR, MPK, LKB1, PAK 1/2/4/5/6, PDGFRA, PYK2, Src, SRPK1, PLC, PKC, PKA, PKB alpha/beta, PKC alpha/gamma/zeta, PKD, PLK1, PRAK, PRK2, WAVE-2, TSC2, DAPKI, BAD, IMP, C-TAK1, TAK1, TAO1, TBK1, TESK1, TGFBR1, TIE2, TLK1, TrkA, TSSK1, TTBK1/2, TTK, Tp12/cotl, MEK1, MEK2, PLDL Erk1, Erk2, Erk5, Erk8, p9ORSK, PEA-15, SRF, p27 KIP1, TIF 1a, HMGN1, ER81, MKP-3, c-Fos, FGF-R1, GCK, GSK3 beta, HER4, HIPK1/2/3/, IGF-1R, cdc25, UBF, LAMTOR2, Statl, StaO, CREB, JAK, Src, PTEN, NF-kappa B, HECTH9, Bax, HSP70, HSP90, Apaf-1, Cyto c, BCL-2, Bcl-xL, Smac, XIAP, Caspase-9, Caspase-3, Caspase-6, Caspase-7, CDC37, TAB, IKK, TRADD, TRAF2, R1P1, FLIP, TAK1, JNK1/2/3, Lck, A-Raf, B-Raf, C-Raf, MOS, MLK1/3, MN 1/2, MSK1, MST2/3/4, MPSK1, MEKKI , ME K4, MEL , ASK1, MINK1 , MKK 1/2/3/4/6/7, NE 2a/6/7, NUAK1, OSR1, SAP , STK33, Syk, Lyn, PDK1, PHK, PIM 1/2/3, Ataxin-1, mTORC1, MDM2, p21 Wafl , Cyclin D1, Lamin A, Tpl2, Myc, catenin, Wnt, IKK-beta, IKK-gamma, IKK-alpha, IKK-epsilon, ELK, p65RelA, IRAKI, IRA 2, IRAK4, IRR, FADD, TRAF6, TRAF3, MKK3, MKK6, ROCK2, RSK1/2, SGK 1, SmMLCK, SIK2/3, ULK1/2, VEGFR1, WNK 1 , YES1, ZAP70, MAP4K3, MAP4K5, MAPKlb, MAPKAP-K2 K3, p38 alpha/beta/delta/gamma MAPK, Aurora A, Aurora B, Aurora C, MCAK, Clip, MAPKAPK, FAK, MARK 1/2/3/4, Mud , SHC, CXCR4, Gap-1, Myc, beta-catenin/TCF, Cbl, BRM, Mcl-1, BRD2, BRD3, BRD4, AR, RAS, ErbB3, EGFR, IRE1, HPK1, RIPK2, and ERct, including all variants, mutations, splice variants, indels and fusions of these target proteins listed.

The B component is a group that binds an E3 ubiquitin ligase. The E3 ubiquitin ligases (of which over 600 are known in humans) confer substrate specificities for ubiquitination. There are known ligands that bind these ligases. As described herein, an E3 ubiquitin ligase binding group may be a peptide or small molecule that can bind an E3 ubiquitin ligase. Examples of E3 ubiquitin ligases include: von Hippel-Lindau (VHL); cereblon, XIAP, E3A; MDM2; Anaphase-promoting complex; UBR5 (EDDI); SOC S/BC-box/eloBC/CUL5/RING; LNXp80; CBX4; CBLL1; HACE1; HECTD1; HECTD2; HECTD3; HECW1; HECW2; HERC1; HERC2; HERC3; HERC4; HUWE1; ITCH; NEDD4; NEDD4L; PPIL2; PRPF19; PIAS1; PIAS2; PIAS3; PIAS4; RANBP2; RNF4; RBX1; SMURF1; SMURF2; STUB1; TOPORS; TRIP12; UBE3A; UBE3B; UBE3C; UBE4A; UBE4B; UBOXS; UBR5; WWP1; WWP2; Parkin; A20/TNFAIP3; AMFR/gp78; ARA54; beta-TrCPl/BTRC; BRCA1; CBL; CHIP/STUB 1; E6; E6AP/UBE3A; F-box protein 15/FBXO15; FBXW7/Cdc4; GRAIL/RNF 128; HOIP/RNF31; cIAP-1/HIAP-2; cIAP-2/HIAP-1; cIAP (pan); ITCH/AIP4; KAP1; MARCH8, Mind Bomb 1/MIB1; Mind Bomb 2/MIB2; MuRF1/TRIM63; NDFIP1; NEDD4; NleL; Parkin; RNF2; RNF4; RNF8; RNF168; RNF43; SART1; Skp2; SMURF2; TRAF-1; TRAF-2; TRAF-3; TRAF-4; TRAF-5; TRAF-6; TRIMS; TRIM21; TRIM32; UBR5; and ZNRF3.

An exemplary E3 ubiquitin ligase is von Hippel-Lindau (VHL) tumor suppressor, which is the substrate recognition subunit of the E3 ligase complex VCB-Cul2. The VCCB-Cul2 complex consists of VHL, elongins B and C, Cul2 and Rbxl. The primary substrate of VHL is Hypoxia Inducible Factor let (HIF-let), a transcription factor that upregulates genes, such as the pro-angiogenic growth factor VEGF and the red blood cell inducing cytokine erythropoietin, in response to low oxygen levels. Compounds that bind VHL may be hydroxyproline compounds, such as those disclosed in WO2013/106643, and other compounds described in US2016/0045607, WO2014187777, US20140356322, and U.S. Pat. No. 9,249,153.

Another exemplary E3 ubiquitin ligase is cereblon. Cereblon is a protein that forms an E3 ubiquitin ligase complex with damaged DNA binding protein 1 (DDB1), Cullin-4A (CUL4A), and regulator of cullins 1 (ROC 1). This complex ubiquitinates a number of other proteins. Cereblon ubiquitination of target proteins results in increased levels of fibroblast growth factor 8 (FGF8) and fibroblast growth factor 10 (FGFIO). FGF8 in turn regulates a number of developmental processes, such as limb and auditory vesicle formation. In the absence of cereblon, DDB1 forms a complex with DDB2 that functions as a DNA damage-binding protein. Thalidomide, lenalidomide, pomalidomide and analogs thereof are known to bind to cereblon. Other small molecule compounds that bind to cereblon are also known, e.g., the compounds disclosed as an in US2016/0058872 and US2015/0291562. Further, phthalimide conjugation with binders, such as antagonists of BET bromodomains can provide PROTACs with highly-selective cereblon-dependent BET protein degradation. Winter et al., Science, Jun. 19, 2015, p. 1376. Such PROTACs can be conjugated to an antibody as described herein to form an APC.

The specificity of a PROTAC relies on its target ligand that binds the target protein for degradation. If the specificity is not high, this can cause off-target effects (side effects). In addition, PROTACs are generally small molecules (MW around 1000). They rely on diffusion to enter cells, which is less efficient (especially with a molecular weight around 1000). Furthermore, because they are small molecules, which typically have short in vivo half-lives due to kidney filtration. Thus, PROTACs may need to be given at a higher dosing frequency.

Antibody-PROTAC conjugates (APCs) of the invention are similar in sizes to antibodies or ADCs, which have long in vivo half-lives. Therefore, APCs of the invention will also have long in vivo half-lives (e.g., weeks) and can enter the cells by internalization due to the presence of antibodies. In addition, APCs have double selectivities: one from antibodies and the other from the target protein binders in PROTACs. For example, the antibody in an APC of the invention may bind a specific antigen on a cancer cell, and then the APC enters the cell via internalization. Once in the cell, the target protein binder in the PROTAC portion finds the target protein and brings it to E3 ubiquitin ligase for ubiquitination. The ubiquitinated target protein is marked for degradation by proteasomes. Thus, an APC of the invention is highly selective and will have less adverse effects.

In addition, APCs of the invention have the advantages of catalytic mode of actions, similar to PROTACs. Therefore, the therapeutically effective doses of APCs can be lower, and they can be given with a lower frequency due to their longer in vivo half-lives. These properties make APCs of the invention more specific and safer to use.

The following Table 1 summarizes and compares some characteristics of ADC, PROTACs, and APCs.

TABLE 1

| | Ideal ADC | Ideal PROTAC | Ideal APC |
|---|---|---|---|
| Molecular | ~150 KD | ~1000 | ~150 KD |
| weight | | | |
| Half life | Long (~week) | Short (~hour) | Long (~week) |
| Administration | i.v. | Oral/i.v. | i.v. |
| Cell penetration | Internalization | Diffusion | Internalization |
| MOA | Payload | Target | Target |
| | | degradation | degradation |
| Efficacious | Low | High | Low |
| dose | | | |
| Therapeutic | Low | Low | High |
| windows | | | |
| Selectivity | From mAb | From target | Double |
| | | ligand | selectivity |
| Toxicity | Payload | Off-target effect | Safer |
| | induced | | |
| | (Stability) | | |

Thus, the APC format is novel and represents a promising approach to new therapeutics. This approach can be generally applied to any target proteins that are associated with any disorders. (see Crews et al., “Proteolysis-Targeting Chimeras: Induced Protein Degradation as a Therapeutic Strategy,” ACS Chem. Biol. 2017, 12(4), 892-898).

Embodiments of the invention can be applied to any target proteins that cause diseases or disorders, by obtaining an antibody and then using the antibody to couple with a PROTAC (i.e., a target protein binder coupled to an E3 ubiquitin ligase ligand or inhibitor). The antibody is directed to an antigen expressed on the cells containing the target protein. The target protein binders in PROTACs will depend on what proteins are being targeted. For example, for a target enzyme (e.g., a kinase), one can design an inhibitor as a ligand/binder.

For E3 ubiquitin ligase ligands/binders, several molecules are known to bind various E3 ubiquitin ligases. Examples include:

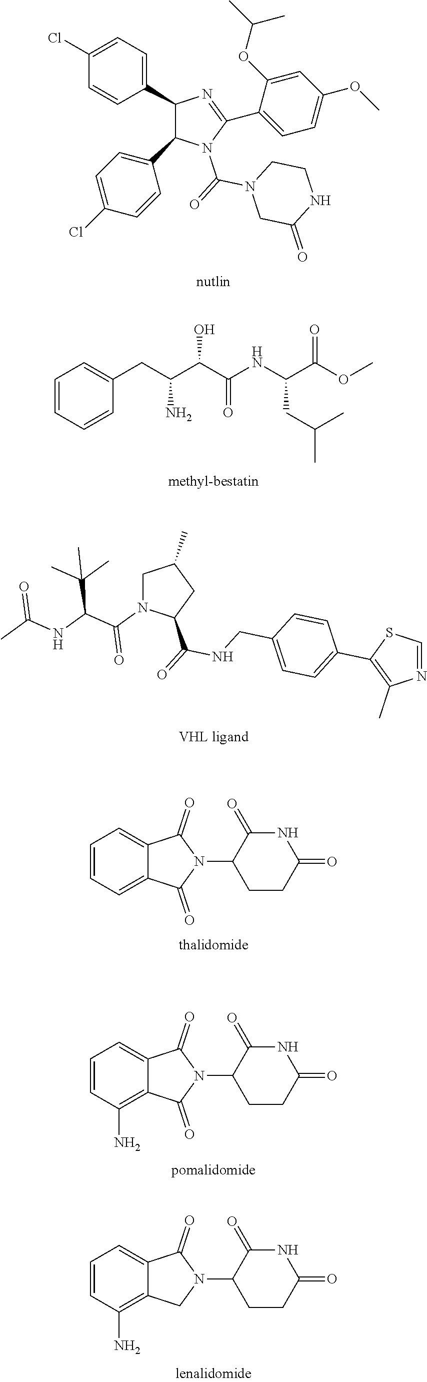

Nutlin derivatives bind MDM2 (double minute 2 homolog; also known as E3 ubiquitin-protein ligase Mdm2), which is a negative regulator of the p53 tumor suppressor. Mdm2 functions as an E3 ubiquitin ligase that recognizes the N-terminal trans-activation domain (TAD) of p53 tumor suppressor and as an inhibitor of p53 transcriptional activation. Bestatin (ubenimex) engages cIAP1 (cellular inhibitor of apoptotic protein-1). The IMiDs thalidomide and its derivatives pomalidomide and lenalidomide bind cereblon.

The following description will use specific examples to illustrate embodiments of the invention. The examples use bromodomain and extra terminal domain (BET) family of proteins as the target proteins, and trastuzumab as the antibody. However, any particular BET inhibitors, ubiquitin ligase 3 inhibitors, and trastuzumab used in these examples are for illustration only and should not be interpreted to limit the scope of the invention. One skilled in the art would appreciate that other modifications and variations are possible without departing from the scope of the invention.

The bromodomain and extra terminal domain (BET) family of proteins, including BRD2, BRD3, and BRD4, play a key role in many cellular processes, including inflammatory gene expression, mitosis, and viral/host interaction by controlling the assembly of histone acetylation-dependent chromatin complexes. Inhibitors of BET proteins reversibly bind the bromodomains or BET proteins: BRD2, BRD3, BRD4, and BRDT. They can prevent protein-protein interactions between BET proteins and acetylated histones and transcription factors. Thus, BET inhibitors have anti-cancer, immunosuppressive, and other effects. The use of BET inhibitors in APCs would target the BET family proteins for ubiquitination, thereby leading to elimination of BET family proteins by proteasomes.

The following describes details of some embodiments of the invention. However, these details are for illustration only, and one skilled in the art would appreciate that other modifications and variations are possible without departing from the scope of the invention.

### EXAMPLE 1

### Preparations of trastuzumab-BRD4-PROTAC-1

### Synthesis of Compound 2

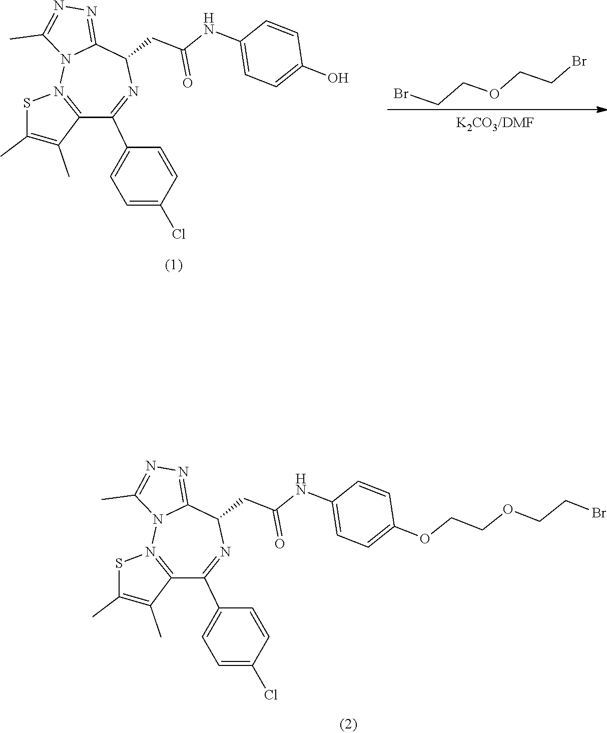

In this example, the BET inhibitor is OTX015, which is an orally bioavailable, small molecule inhibitor of BRD2, BRD3, and BRD4 (EC₅₀=10−19 nM). OTX015 down-regulates c-Myc expression and induces cell cycle arrest and apoptosis. Thus, it has antiproliferative effects against a variety of solid tumors and leukemias.

Compound 2: To a mixture of OTX015 (1) (0.2 mmol) and 1-bromo-2-(2-bromoethoxy)ethane (lmmol) in dimethylformamide (5 mL) was added potassium carbonate (0.6 mmol). The mixture was stirred for 24 hours at 50° C. After the reaction was complete, the reaction mixture was extracted with dichloromethane and water. Then, the organic layer was washed with brine and dried over MgSO₄. The organic solvent was removed under reduced pressure. The residue was purified by column chromatography with methanol:dichloromethane (1:19) to afford a yellow solid Compound 2 (58% yield). ¹H NMR (600 MHz, chloroform-d): δ 7.45 (d, J=9.1 Hz, 2H), 7.38 (d, J=8.6 Hz, 2H), 7.28 (d, J=8.6 Hz, 2H), 6.78 (d, J=9.1 Hz, 2H), 4.71 (dd, J=8.0, 6.2 Hz, 1H), 4.06 (dd, J=5.4, 4.5 Hz, 2H), 3.86-3.82 (m, 4H), 3.78 (dd, J=14.5, 8.0 Hz, 1H), 3.57 (dd, J=14.5, 6.2 Hz, 1H), 3.47 (t, J=6.2 Hz, 2H), 2.66 (s, 3H), 2.38 (d, J=0.6 Hz, 3H), 1.65 (d, J=0.6 Hz, 3H). LCMS (ESI): m/z Calcd for [C₃₃H₃₇ClN₆O₄S+H]⁺ 642.08, found 642.52 [M+H]⁺.

### Synthesis of Comnound 4

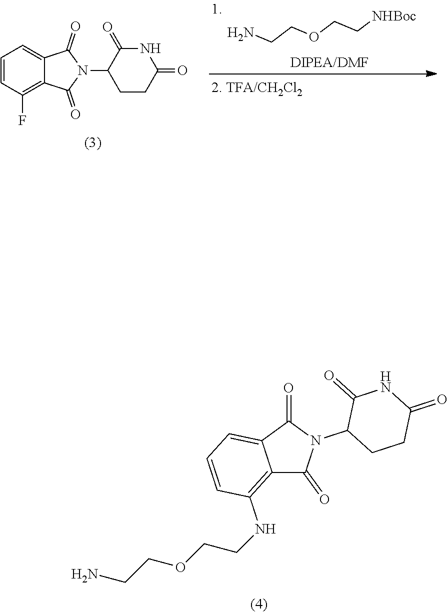 [SMILES: O=C1CCC(N2C(=O)c3cccc(F)c3C2=O)C(=O)N1 ; CC(C)(C)OC(=O)NCCOCCN ; NCCOCCNc1cccc2c1C(=O)N(C1CCC(=O)NC1=O)C2=O]

Compound 4: To a solution of pomalidomide (3) (0.2 mmol) and tert-butyl (2-(2-aminoethoxy)ethyl) carbamate (0.22 mmol) in DMF (5 mL) was added N,N-diisopropylethylamine (0.4 mmol). The reaction mixture was stirred at 90° C. for 12 hours. After the reaction was complete, the reaction mixture was extracted with dichloromethane and water. Then, the organic layer was washed with brine and dried over MgSO₄.The organic solvent was removed under reduced pressure. After purification on a flash column with ethyl acetate:hexane (2:3), the yellow residue was dissolved in dichloromethane and trifluoroacetic acid (1 mL) was added. The mixture was stirred at room temperature for 0.5 hours. Afterwards, the reaction mixture was extracted with dichloromethane and water. Then, the organic layer was washed with brine and dried over MgSO₄.The organic solvent was removed under reduced pressure. The residue was purified by column chromatography with methanol:dichloromethane (1:9) to afford a yellow solid Compound 4. ¹H NMR (600 MHz, chloroform-d) δ 7.35 (t, J=7.8 Hz, 1H), 6.93 (d, J=7.0 Hz, 1H), 6.78 (d, J=8.6 Hz, 1H), 6.38 (d, J=5.1 Hz, 1H), 4.81 (dd, J=11.6, 5.1 Hz, 1H), 3.62 (d, J=4.0 Hz, 2H), 3.56 (d, J=4.5 Hz, 2H), 3.33 (d, J=4.1 Hz, 2H), 3.05-3.04 (m, 2H), 2.71-2.53 (m, 3H), 2.01-1.93 (m, 1H). LCMS (ESI): m/z Calcd for [C₃₃H₃₇ClN₆O₄S+H]⁺ 361.14, found 361.22 [M+H]⁺.

### Synthesis of Compound 5

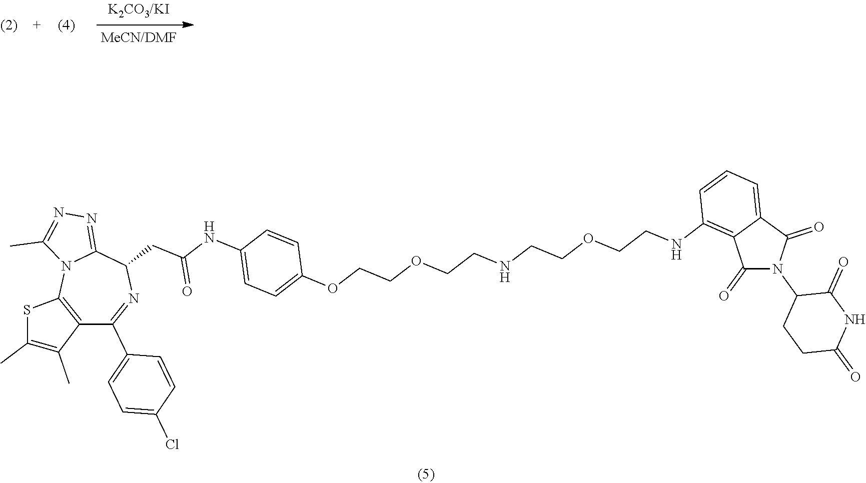 [SMILES: Cc1sc2c(c1C)C(c1ccc(Cl)cc1)=N[C@@H](CC(=O)Nc1ccc(OCCOCCNCCOCCNc3cccc4c3C(=O)N(C3CCC(=O)NC3=O)C4=O)cc1)c1nnc(C)n1-2]

Compound 5: To a solution of Compound 2 (0.2 mmol), Compound 4 (0.6 mmol) and potassium iodide (0.2 mmol) in acetonitrile/dimethylformamide (3:1, 4 mL) was added potassium carbonate (0.6 mmol). The reaction mixture was stirred at 50° C. for 72 hours. After the reaction was complete, the reaction mixture was extracted with dichloromethane and water. Then, the organic layer was washed with brine and dried over MgSO₄.The organic solvent was removed under reduced pressure. The residue was purified by column chromatography with methanol:dichloromethane (1:12) to afford a yellow solid Compound 5 (19.7% yield). ¹H NMR (600 MHz, chloroform-d) δ 7.53-7.36 (m, 5H), 7.36-7.29 (m, 2H), 7.09 (dd, J=7.0, 3.7 Hz, 1H), 6.88 (d, J=8.7 Hz, 1H), 6.82 (dd, J=8.7, 1.4 Hz, 2H), 4.89 (dt, J=12.5, 6.1 Hz, 1H), 4.66 (ddd, J=7.9, 6.0, 3.5 Hz, 1H), 4.10-4.02 (m, 2H), 3.84-3.58 (m, 9H), 3.55-3.41 (m, 2H), 3.41-3.30 (m, 2H), 3.02-2.91 (m, 3H), 2.87-2.68 (m, 3H), 2.67 (s, 3H), 2.40 (s, 3H), 2.10-2.07 (m, 1H), 1.67 (s, 3H). LCMS (ESI): m/z Calcd for [C₃₃H₃₇ClN₆O₄S+H]⁺922.30, found 922.49 [M+H]⁺

### Synthesis of Compound 7

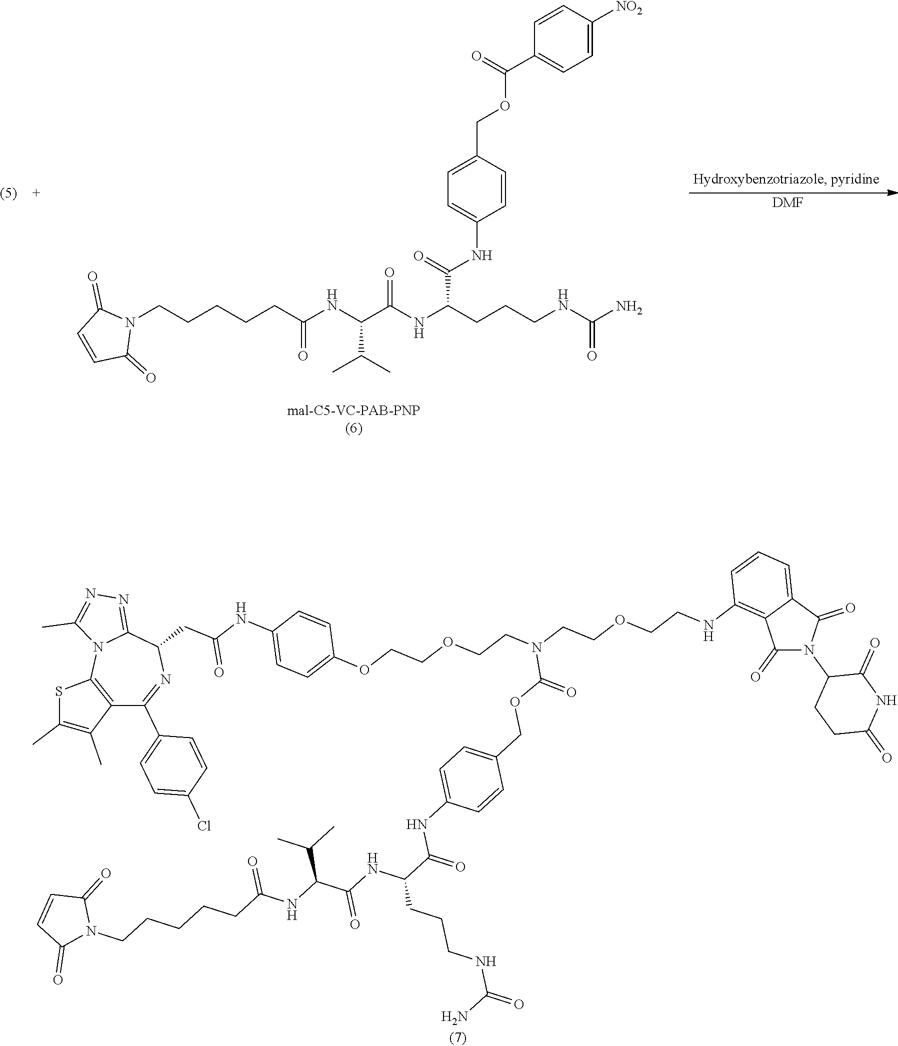

Compound 7: To a solution of Compound 5 (0.2 mmol) and Mal-05-VC-PAB-PNP Compound 6 (0.2 mmol) in dimethylformamide (5 mL) was added hydroxybenzotriazole (0.4 mmol) and pyridine (0.4 mmol). The reaction mixture was stirred at room temperature for 72 hours. After the reaction was complete, the reaction mixture was extracted with dichloromethane and water. Then, the organic layer was washed with brine and dried over MgSO₄.The organic solvent was removed under reduced pressure. The residue was purified by column chromatography with methanol:dichloromethane (1:16) to afford a yellow solid Compound 7 (46.3% yield). ¹H NMR (600 MHz, chloroform-d) δ 7.55-7.40 (m, 7H), 7.33 (d, J=7.2 Hz, 2H), 7.25-7.19 (m, 2H), 7.08 (d, J=7.0 Hz, 1H), 6.92-6.83 (m, 1H), 6.79-6.68 (m, 2H), 6.68-6.65 (m, 2H), 5.07-5.00 (m, 2H), 4.77-4.71 (m, 1H), 4.71-4.60 (m, 1H), 4.37-4.29 (m, 1H), 4.04-3.96 (m, 1H), 3.93-3.85 (m, 1H), 3.77-3.34 (m, 21H), 3.28-3.14 (m, 1H), 3.11-3.02 (m, 1H), 2.93-2.71 (m, 3H), 2.69 (s, 3H), 2.42 (s, 3H), 2.04-2.01 (m, 1H), 1.91-1.81 (m, 2H), 1.77-1.70 (m, 1H), 1.69 (s, 3H), 1.62-1.54 (m, 4H), 1.33-1.25 (m, 5H), 0.91-0.87 (m, 6H). LCMS (ESI): m/z Calcd for [C₃₃H₃₇ClN₆O₄S+H]⁺ 1520.58, found 1521.14 [M+H]⁺.

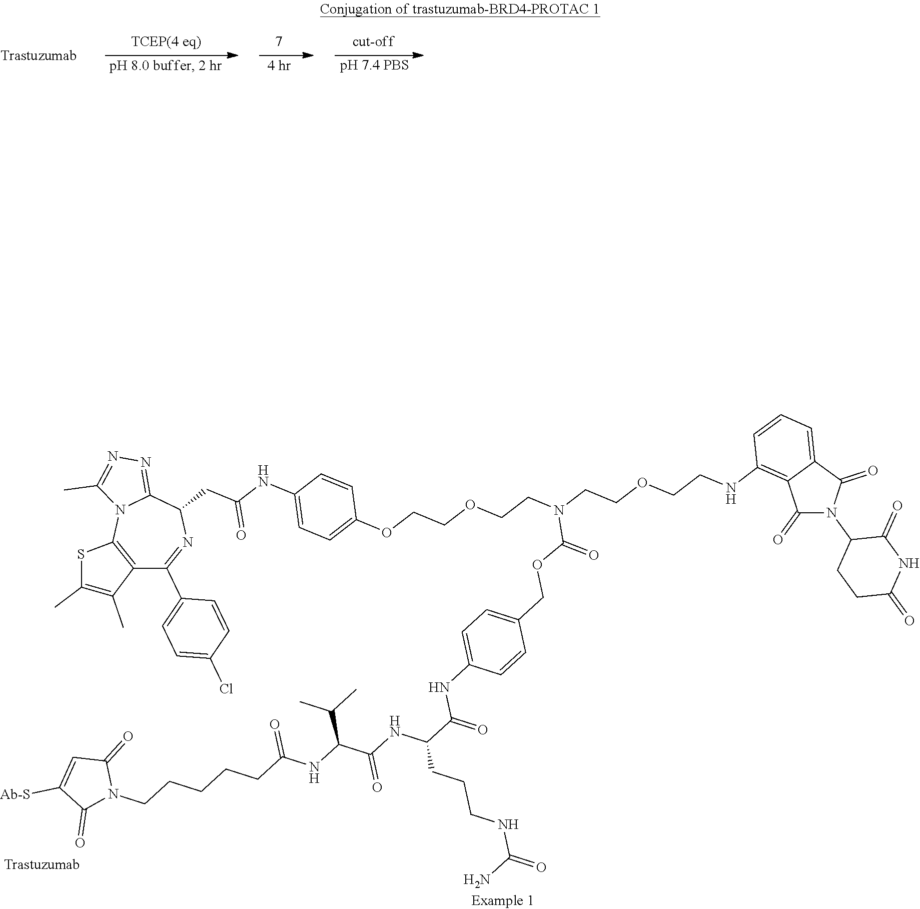

To a solution of trastuzumab 1 mg (5.0 mg/mL) in buffer (25 mM sodium borate pH 8, 0.025 M NaCl, 1 mM diethylenetriaminepentaacetic acid (DTPA)) was treated with tris(2-carboxyethyl)phosphine (TCEP, 4.0 molar equiv) at 37° C. for 2 hours. The excess TCEP was removed using an Amicon Ultra-15 centrifugal filter device with 30 kDa NMWL in buffer (25 mM sodium borate pH 8, 0.025 M NaCl, 1 mM DTPA) and then treated with Compound 7 (20 molar equiv) at 25° C. for 4 hours. The reaction mixture was cut-off and concentrated by using an Amicon Ultra-15 centrifugal filter device with 30 kDa NMWL in pH 7.4 PBS buffer to give trastuzumab-BRD4-PROTAC 1.

### EXAMPLE 2

### Preparations of trastuzumab-BRD4-PROTAC-2

### Synthesis of Compound 8

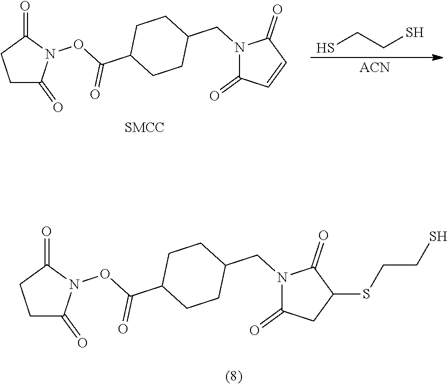

Compound 8: To a solution of succinimidyl 4-(N-maleimidomethyl) cyclohexane-1-carboxylate (SMCC) (0.75 mmol) in acetonitrile (7 mL) was added 1,2-ethanedithiol (0.82 mmol). The reaction mixture was stirred at room temperature for 3 hours. After the reaction was complete, the reaction mixture was extracted with dichloromethane and water. Then, the organic layer was washed with brine and dried over MgSO₄.The organic solvent was removed under reduced pressure. The residue was purified by column chromatography with ethyl acetate:hexane (3:2) to afford a white solid Compound 8 (34.8% yield).

### Synthesis of Compound 9

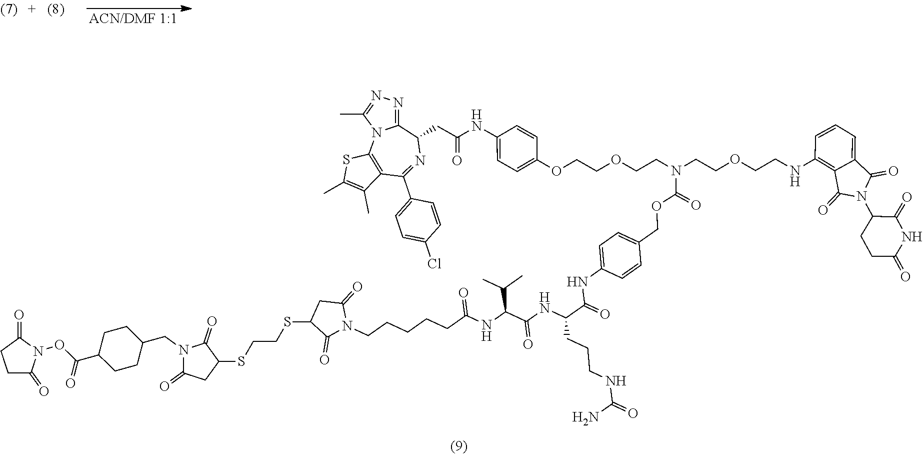 [SMILES: "ACN/DMF 1:1" ; Cc1sc2c(c1C)C(c1ccc(Cl)cc1)=N[C@@H](CC(=O)Nc1ccc(OCCOCCN(CCOCCNc3cccc4c3C(=O)N(C3CCC(=O)NC3=O)C4=O)C(=O)OCc3ccc(NC(=O)[C@H](CCCNC(N)=O)NC(=O)[C@@H](NC(=O)CCCCCN4C(=O)CC(SCCSC5CC(=O)N(CC6CCC(C(=O)ON7C(=O)CCC7=O)CC6)C5=O)C4=O)C(C)C)cc3)cc1)c1nnc(C)n1-2]

Compound 9: To a solution of Compound 7 (0.02 mmol) in acetonitrile/dimethylformamide (1:1, 6 mL) was added Compound 8 (0.04 mmol). The reaction mixture was stirred at room temperature for 16 hours. After the reaction was complete, the reaction mixture was extracted with dichloromethane and water. Then, the organic layer was washed with brine and dried over MgSO₄.The organic solvent was removed under reduced pressure. The residue was purified by column chromatography with ethyl acetate:hexane (3:2) to afford a yellow solid Compound 9 (86.2% yield).

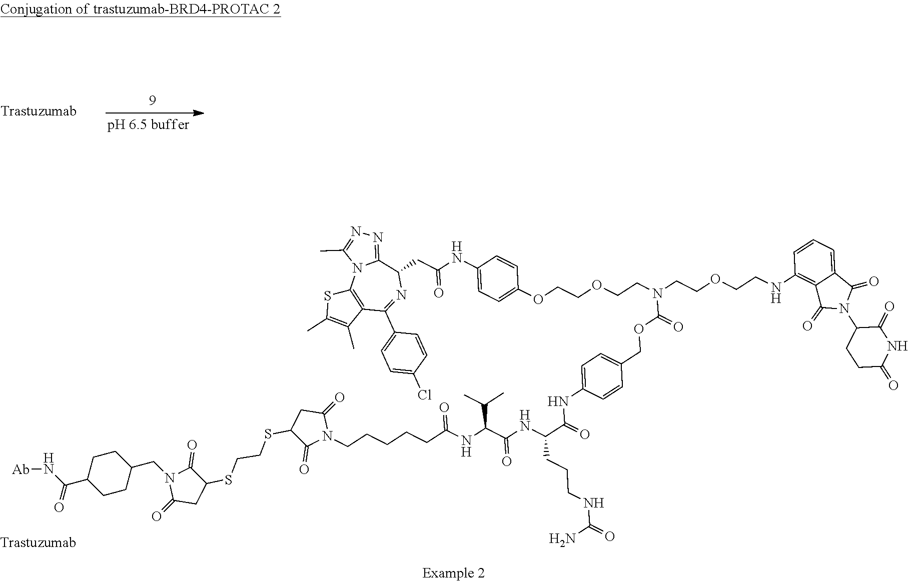

To a solution of trastuzumab 1 mg (5.0 mg/mL) in buffer (50 mM potassium phosphate, 50 mM sodium chloride, 2 mM EDTA; pH 6.5) was slowly added 30 equivalents of 9 (5 mM in DMSO). The reaction mixture was stirred at 37° C. for 18 hours. Desalt and concentrate the antibody preparation using an Amicon Ultra-15 centrifugal filter device with 30 kDa NMWL in pH 7.4 PBS buffer to give trastuzumab-BRD4-PROTAC 2.

As noted above, an APC of the invention has a branched form, in which the antibody-L² linker is attached the L¹ linker in a PROTAC. As illustrated in the above examples, attachments of the L² linker to the L¹ linker would not alter the A binders or B binders of the PROTACs and the attachment reactions are relatively easy. As a comparison, the following example illustrates an attempt to synthesize a linear (non-branched) APC, in which L² linker is coupled to the A binder or the B binder.

### EXAMPLE 3

### Synthesis of BRD4-PROTAC with Linear Form (17)

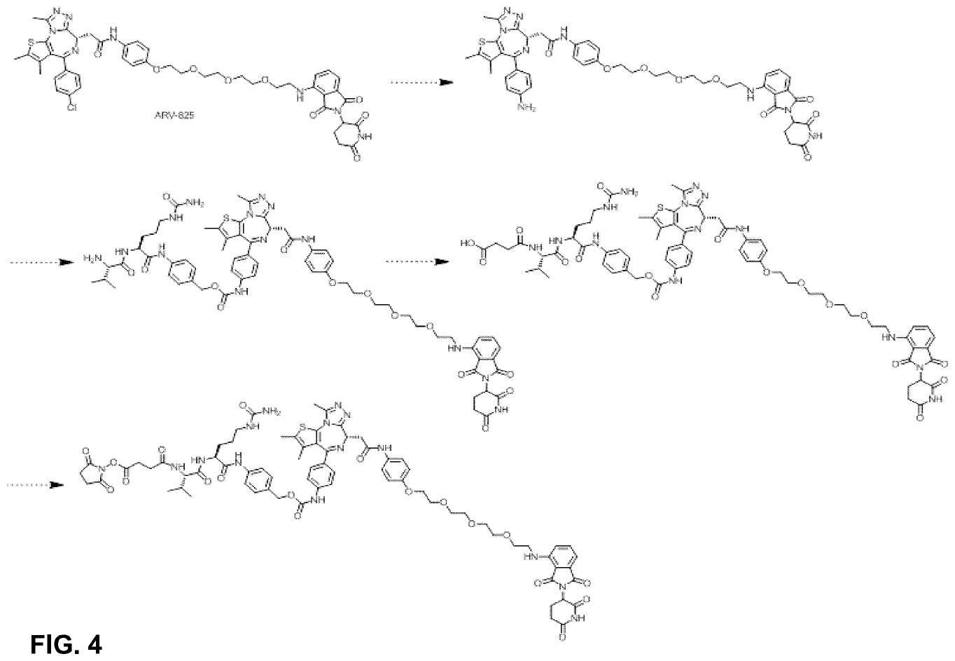

FIG. 4 illustrates a synthetic scheme for possible synthesis of linkage via the A binder of ARV-825. The functional group modification of the protein ligand or ligase binder is not easy. Furthermore, not all protein ligands or ligase binders have suitable function groups for modifications. In this example, the chloride atom on the OTX015, the protein ligand of PROTAC ARV-825, is difficult to transform to another functional group, such as an amino group. OTX015 or ARV-825 shows no reactivity under Buchwald reaction (palladium catalyzed coupling reactions) and Ullman reaction (copper catalyzed coupling reactions). Harsh reaction conditions, such as metal halide exchange, will lead to compound decomposition. According to the literature (EP1887008A1), different functional group of BRD4 inhibitors should be introduced at the very beginning. In other words, directly coupling the linker with the protein ligand or ligase binder will make the synthesis more complicated.

In contrast, the branched linker strategy as disclosed in this invention affords a new method of connecting any protein ligand or ligase binder to form APCs with the following advantages. The APCs maintain the structures of the target protein ligands and E3 ligase ligands, and therefore the binding affinities will not change. Structural modifications for attaching groups on linkers are much easier than on ligands. Modifications on linkers are suitable for most PROTACs and one can design a “common” coupling functional group for different PROTACs such that the same antibody can be coupled with different PROTACs or the same PROTAC can be coupled with different antibodies.

### EXAMPLE 4

### Biological Activity

Various immunoconjugates of Formula (I) were tested for their specificity and abilities for degrading targeting protein. Brief descriptions of different assays are described below.

### Western Blot

Evaluate the cellular potencies of compounds of Formula (I) in BRD4 protein degradation. Bromodomain protein 4 (BRD4) is one of the BET (bromodomain and extra-terminal) family proteins and is implicated in tumorigenesis of hematological malignancies and solid tumors. BRD4 recognizes and binds acetylated histones and plays a key role in transmission of epigenetic memory across cell divisions and transcription regulation. Potent inhibitors targeting BRD4 display anti-tumor activities, suppressing the proliferation and transformation of various cancer cells. This led to BRD4 as a promising therapeutic target for cancer treatment. BRD4 proteolysis targeting chimera (PROTAC) have been shown to possess anticancer activities by inducing BRD4 protein degradation. However, in addition to the anti-cancer effects, normal cells are also affected by these agents. This leads to concerning adverse effects of BET inhibition, such as autism-like syndrome and impairment in memory formation.

In the current invention, a BRD4-PROTAC antibody conjugate with the branched format was created in order to keep intact the BRD4-PROTAC modality, enhance the specificity of cancer cell targeting, and reduce the potential off-target effects. “BRD4-PROTAC” refers to a PROTAC that includes a target binder for the BRD4 protein. By coupling “BRD4-PROTAC” with an antibody that recognizes an antigen on cancer cells. The high specificity of the antibody allows the resulting conjugate (i.e., Ab-BRD4-PROTAC) to target specific cancer cells and cause less toxicity to healthy cells. For example, the antibody may be trastuzumab and the cancer cells may be HER2-positive BT-474 breast cancer cells. Various compounds of Formula (I) (i.e., with different BRD4 binders, different E3 binders, and/or different antibodies) were tested in cellular BRD4 protein degradation via western blotting assay. The following uses trastuzumab-coupled BRD4-PROTACs to illustrate the benefits of embodiments of the invention.

For western blot experiments, HER2-positive BT-474 and HER2-negative MDA-MB-231 breast cancer cells were cultured in DMEM and L15 medium with 10% FBS, respectively, and cultured overnight. On the assay day, two hundred thousand cells were pretreated with each of the test compounds for 24 hours. After 24 hours, the whole cell lysate was harvested by adding 2× SDS Sample Buffer. Proteins were separated by SDS-PAGE electrophoresis and transfer to PVDF membrane. Protein expression was detected using immunoblot with various primary antibodies and secondary antibodies following standard protocols. Antibody against BRD4 and anti-rabbit IgG, HRP-linked secondary antibodies were purchased from Cell Signaling Technology (Danvers, Mass.). Antibody against actin was purchased from Millipore (Burlington, Mass.). Immunoblots were revealed by chemiluminescence (SuperSignal™ West Femto Maximum Sensitivity Substrate, Thermo Fisher, Waltham, Mass.) and detected by ChemiDoc™ MP Imaging System (Bio-Rad, Hercules, Calif.). Band intensities of western blot were also quantified by ChemiDoc™ MP Imaging System. Relative intensities of bands corresponding to the drug treatment group were compared to those of the untreated group.

FIG. 2 shows results of the assay. ARV-825 (CAS#1818885-28-7) is a hetero-bifunctional molecule comprising a BRD4 binding moiety linked to an E3 ligase cereblon binding moiety. ARV-825 is a proteolysis targeting chimera (PROTAC). (see Lu, J. et al., “Hijacking the E3 ubiquitin ligase cereblon to efficiently target BRD4,” Chem. Biol. 22(6), 755-763 (2015)). Inventive Compound 5 is a branch form of ARV-825. Inventive Compound 7 is a Compound 5 with an additional lysosomally cleavable dipeptide (Valine-Citrulline) linker.

As shown in FIG. 2, Compound 5 is as effective as ARV-825 in promoting the degradation of BRD4 in this cell culture assay. In contrast, Compound 7 cannot degrade BRD4 protein up to 1 μM of Compound 7 treatment which possibly resulted from Valine-Citrulline dipeptide leading lower permeability. This result suggests that if an APC gets prematurely cleaved before entry into cells, the released PROTAC (e.g., Compound 7) would not cause off-target effects. Thus, APCs of the invention would have higher safety margins.

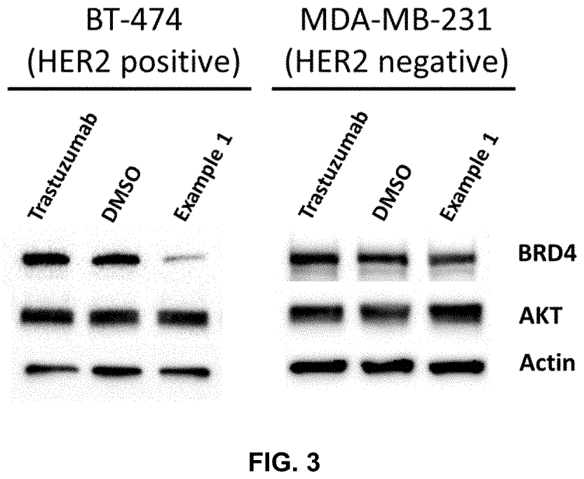

FIG. 3 shows that Compound 7 trastuzumab antibody conjugate (i.e., example 1) exhibits a specific BRD4 protein degradation activity in HER2-positive BT-474 breast cancer cells instead of HER2-negative MDA-MB-231 breast cancer cells. This APC would not cause degradation of either AKT protein or actin protein. This indicates that the branched coupling of the antibody to the linker in a PROTAC did not compromise the activity of the PROTAC. Importantly, under the in vivo situations, the antibody (trastuzumab) would direct the APC to those cells expressing the specific antigen (e.g., HER2), thereby reducing the off-target effects. Accordingly, example 1 would be as effective but safer than the AV-825 in clinical applications.

These results indicated that the antibody conjugates of the invention (branched APCs) can enhance the specificity of cancer cell targeting, enforce the cellular uptake of PROTAC modality, and reduce the potential off-target effects. In addition, if the antibody is separated prematurely, the released PROTACs (e.g., Compound 7) have relatively lower permeabilities due to the Valine-Citrulline dipeptide and would not cause the undesired off-target effects, leading to a high safety margin.

### Anti-Proliferation Activity

As noted above, APCs of the invention may be used to treat diseases or disorders harboring specific antigens. The diseases may be cancers, autoimmune diseases, infectious diseases, or blood vessel proliferative disorders. The cancers may be lung cancer, colon cancer, colorectal cancer, breast cancer, prostate cancer, liver cancer, pancreatic cancer, bladder cancer, gastric cancer, renal cancer, salivary gland cancer, ovarian cancer, uterine body cancer, cervical cancer, oral cancer, skin cancer, brain cancer, lymphoma, or leukemia. Inhibition of cell growths by APCs of the invention were measured using CellTiter™-96 assay. The cytotoxicities of APCs were evaluated in breast cancer cell lines with different HER2 expression phenotypes. The results show that APCs of the present invention are only toxic to HER2-positive breast cancer cells, wherein BRD4 protein, Src kinase, or RAS protein may be specifically targeted for proteasome degradation by using proper PROTACs coupled to trastuzumab.

The APCs of the invention are promising new therapeutics because they have the advantages of ADCs and PROTACs. Moreover, the branched APCs of the invention show advantages over linear ADC PROACs and are useful in treating disorders harboring specific antigens.

Some embodiments of the invention relate to methods of treating a disease or disorder using an APC of the invention. The disease may be a cancer. Specific examples of cancers may include breast cancer, gastric cancer, squamous cell carcinoma, colon cancer, and leukemia expressing specific antigen. The antibody used in APCs may be trastuzumab, cetuximab, rituximab, brentuximab, gemtuzumab, inotuzumab, sacituzumab, alemtuzumab, nimotuzumab. Specific examples of APCs may be branched trastuzumab-coupled PROTACs for targeting breast cancer or gastric cancer with HER2 expression.

The branched antibody-coupled PROTACs can be synthesized in different format such as different linker or different antibody conjugation method. Compound 18 and Compound 19 are examples which shows different linker forms for lysine conjugation. The synthesis of the PROTACs in Compound 18 and Compound 19 follow the same process as Compound 9 with PEG linkers. The Compound 18 and Compound 19 can be synthesized in the same process as example 2 described above. The branched PROTAC with PEGs linker can perform better solubility and conjugation with antibody.

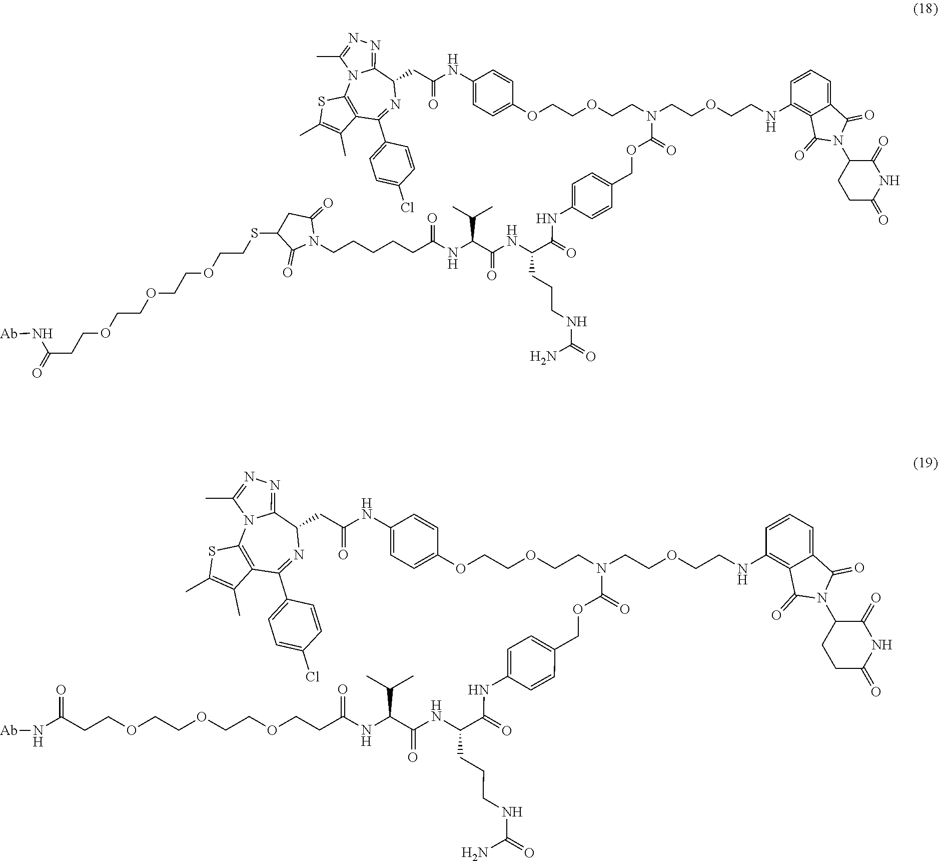

Embodiments of the invention have been illustrated with a limited number of examples. One skilled in the art would appreciate that other modifications and variations are possible without departing from the scope of the invention. Therefore, the scope of protection should only be limited by the attached claims.

What is claimed is:

1. An immunoconjugate having the structure of Formula (I), 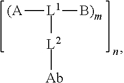
wherein:
(a) Ab is an antibody or a binding fragment thereof;
(b) L¹ and L² are each independently a linker, wherein L¹ and L² are the same or different, and wherein L¹ links to L²;
(c) A is a target-protein ligand/binder;
(d) B is a ubiquitin ligase ligand/binder, and
(e) n and m are each independently an integer from 1 to 8.

2. The immunoconjugate of claim 1, wherein the target protein is selected from the group consisting of a kinase, a G protein-coupled receptor, a transcription factor, a phosphatase, and a RAS superfamily member.

3. The immunoconjugate of claim 1, wherein A is selected from the group consisting of a eat Shock Protein 90 (HSP90) inhibitor, a Kinase or Phosphatase inhibitor, an MDM2 inhibitor, an HDAC inhibitor, a Human Lysine Methyltransferase Inhibitor, an angiogenesis inhibitor, an immunosuppressive compound, and a compound that targets: Human BET Bromodomain-containing proteins, the aryl hydrocarbon receptor (AHR), REF receptor kinase, FKBP, Androgen Receptor (AR), Estrogen receptor (ER), Thyroid Hormone Receptor, HIV Protease, HIV Integrase, HCV Protease, or Acyl-protein Thioesterase-1 and -2 (APT1 and APT2).

4. The immunoconjugate of claim 1, wherein B is a group that binds an E3 ligase selected from the group consisting of XIAP, VHL, cereblon, and MDM2.

5. The immunoconjugate of claim 1, where Ab is a monoclonal antibody or a variant thereof.

6. The immunoconjugate of claim 1, wherein Ab binds to one or more of polypeptides selected from the group consisting of DLL3, EDAR, CLL1; BMPR1B; E16; STEAP1; 0772P; MPF; NaPi2b; Sema 5b; PSCA hlg; ETBR; MSG783; STEAP2; TrpM4; CRIPTO; CD21; CD79b; FcRH2; B7-H4; HER2; NCA; MDP; IL20Rct; Brevican; EphB2R; ASLG659; PSCA; GEDA; BAFF-R; CD22; CD79a; CXCRS; HLA-DOB; P2X5; CD72; LY64; FcRH1; IRTA2; TENB2; PMEL17; TMEFF1; GDNF-Ra1; Ly6E; TMEM46; Ly6G6D; LGR5; RET; LY6K; GPR19; GPR54; ASPHD1; Tyrosinase; TMEM118; GPR172A; MUC16, and CD33.

7. The immunoconjugate according to claim 5, wherein Ab is trastuzumab, cetuximab, rituximab, brentuximab, gemtuzumab, inotuzumab, sacituzumab, alemtuzumab, or nimotuzumab.

8. A pharmaceutical composition comprising the immunoconjugate of claim 1 and one or more pharmaceutically acceptable excipients.

9. A method for treating a disease, comprising: administrating to a subject in need thereof a pharmaceutical composition that comprises an effective amount of the immunoconjugate of claim 1.

10. The method according to claim 9, wherein the disease is a cancer.

11. The method according to claim 10, wherein the cancer is breast cancer or gastric cancer, and the Ab is trastuzumab

12. The method according to claim 10, wherein the cancer is colon cancer or squamous cell carcinomas and the Ab is cetuximab.

13. The method according to claim 10, wherein the disease is leukemia and the Ab is rituximab, brentuximab, gemtuzumab, inotuzumab, or alemtuzumab.
